(19) United States
(12) Patent Application Publication
(10) Pub. No.: US 2024/0206869 A1
(43) Pub. Date: Jun. 27, 2024

(54) DEVICES, SYSTEMS, AND METHODS FOR PERFORMING SUTURING PROCEDURES

(71) Applicant: INTUITIVE SURGICAL OPERATIONS, INC., Sunnyvale, CA (US)

(72) Inventor: Ronald G. Litke, Sandy Hook, CT (US)

(73) Assignee: INTUITIVE SURGICAL OPERATIONS, INC., Sunnyvale, CA (US)

(21) Appl. No.: 18/553,058

(22) PCT Filed: Mar. 28, 2022
(86) PCT No.: PCT/US22/22153

§ 371 Date: Sep. 28, 2023

Related U.S. Application Data
(60) Provisional application No. 63/167,274, filed on Mar. 29, 2021.

(51) Int. Cl. A61B 17/04; A61B 17/00; A61B 34/35; A61B 34/37

(52) U.S. Cl. CPC A61B 17/0469; A61B 17/0482; A61B 34/35; A61B 34/37; A61B 2017/00017; A61B 2017/00398

(57) ABSTRACT

A needle driver device comprises a needle track defining a curved path and a drive system configured to operably engage with and drive a needle along the needle track. The drive system comprises a first drive member, a second drive member, and a needle driver link. The first drive member is rotatable about a first drive member axis, and the second drive member is rotatable about a second drive member axis spaced apart from the first drive member axis. The needle driver link comprises a distal end portion removably engageable with a needle that is positionable in the needle track, the distal end portion being movable along an arcuate-shaped path proximate the needle track. Devices, systems, and methods relate to needle driver devices.

### TECHNICAL FIELD

Aspects of the present disclosure relate to devices, systems, and methods for performing a suturing procedure. For example, aspects of the present disclosure relate to needle driver devices including, but not limited to, for example, devices configured to insert sutures during remote surgical, diagnostic, therapeutic, and other medical procedures. Further aspects of the disclosure relate to methods of operating such devices.

### INTRODUCTION

Sutures are used in a variety of medical applications, such as closing ruptured or incised tissue, soft tissue attachment, attachment of grafts, etc. Additionally, sutures may have other medical and/or non-medical uses. Conventionally, suturing is accomplished by penetrating tissue with the sharpened tip of a suturing needle that has a thread of suturing material attached to the opposite blunt end of the needle. The needle is then pulled through the tissue, causing the attached thread of suturing material to follow the path of the needle. Typically, a knot is tied at the trailing end of the thread to anchor the first stitch. This action is performed repetitively with application of tension to the needle to pull a length of the thread through the tissue using subsequent stitches until the tissue is sutured as desired with one or more stitches. At the conclusion of a suturing procedure, any excess amount of suturing material may be trimmed from the amount remaining after the knot at the trailing end.

While the above-described suturing process can be performed manually, automated suturing systems have also been developed. For example, some systems include a needle driver device configured to draw suturing material through tissue segments, similar to the manual suturing procedure described above.

It is desirable when performing certain suturing procedures to provide needle driver devices that occupy a minimal amount of space relative to a size (e.g., gauge and/or radius) of the needle. Such needle-drive devices are useful in space-limited applications, such as in the case of minimally invasive medical procedures, for example laparoscopic surgery or computer-assisted surgery.

A need exists to provide needle driver devices with an overall relatively small working end. A need also exists to provide robust mechanical parts and operational design of such devices to reduce complexity and/or wear on parts of the device, to provide consistent operational performance, and to increase efficiency of suturing operations.

### SUMMARY

Embodiments of the present disclosure may solve one or more of the above-mentioned problems and/or may demonstrate one or more of the above-mentioned desirable features. Other features and/or advantages may become apparent from the description that follows.

In accordance with at least one aspect of the present disclosure, a needle driver device comprises a needle track defining a curved path and a drive system configured to operably engage with and drive a needle along the needle track. The drive system comprises a first drive member, a second drive member, and a needle driver link. The first drive member is rotatable about a first drive member axis and the second drive member is rotatable about a second drive member axis spaced apart from the first drive member axis. The needle driver link comprises a distal end portion removably engageable with a needle that is positionable in the needle track, the distal end portion being movable along an arcuate-shaped path proximate the needle track, a first portion rotatably coupled to the first drive member at a location offset from the first drive member axis by a first distance, and a second portion between the first portion and the distal end portion, the second portion rotatably coupled to the second drive member at a location offset from the second drive member axis by a second distance different from the first distance.

In accordance with at least another aspect of the present disclosure, a needle driver device comprises a curved needle track defining a curved path, a rotary drive member, and a needle driver link. The needle driver link comprises a distal end portion removably engageable with a curved needle configured to be received in the curved needle track, a first portion coupled to the rotary drive member, and a longitudinal axis extending between the distal end portion and the first portion. The needle driver link moves in response to rotation of the rotary drive member to drive the curved needle along the curved needle track, and the distal end portion traverses an arcuate-shaped path that deviates in shape from an arcuate-shaped path along which the curved needle moves along the curved needle track.

In yet another aspect of the present disclosure, a method of operating a needle driver device comprises rotating a drive member coupled to a first portion of needle driver link, moving a distal end portion of the needle driver link in response to rotating the drive member, and moving a curved needle along a curved needle track in response to moving the distal end portion of the needle driver link. The distal end portion traverses an arcuate-shaped path that deviates in shape from an arcuate-shaped path along which the curved needle moves along the curved needle track.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are for example and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings,

FIG. 1 is a schematic, side perspective view of an embodiment of a needle driver device according to some embodiments of the present disclosure.

FIG. 2 is a top, interior view of a distal portion of some embodiments of a needle driver device.

FIG. 3 is a perspective view of some embodiments of a needle driver device.

FIG. 4 is a perspective, interior view of the distal portion of the needle driver device of FIG. 3.

FIG. 5 is a plan, interior view of the distal portion of the needle driver device of FIG. 4 taken from the side of the needle drive device depicted in FIG. 4.

FIG. 6 is a plan view of various internal components of the needle driver device of FIG. 4 from the side of the needle drive device opposite to that depicted in FIGS. 4 and 5.

FIG. 7 is a graphical representation of a path of a distal end of a driver link of a needle driver device according to various embodiments of the present disclosure.

FIG. 8 is a perspective view of drive components of a needle driver device according to some embodiments of the present disclosure.

FIG. 9 is a perspective view of a manipulator system according to some embodiments of the disclosure.

FIG. 10 is a partial schematic view of an embodiment of a manipulator system having a manipulator arm with two instruments in an installed position according to some embodiments of the present disclosure.

### DETAILED DESCRIPTION

Automated suturing systems can have particular application in conjunction with minimally-invasive surgical procedures. Such procedures may involve the use of remotely-controlled surgical instruments including, for example, teleoperated surgical instruments (e.g., surgical instruments operated at least in part with computer assistance, such as instruments operated with robotic technology) as well as manually operated (e.g., laparoscopic, thoracoscopic) surgical instruments. During such procedures, a surgical instrument, which may extend through a cannula inserted into a patient's body, can be remotely manipulated to perform a procedure at a surgical site. For example, in a teleoperated surgical system, cannulas and surgical instruments can be mounted at manipulator arms of a patient side cart and can be remotely manipulated via teleoperation at a surgeon console.

The present disclosure provides various embodiments of needle driver devices, and related systems and methods, that include features that contribute to a low overall size of the needle driver device, such as a relatively small outer diameter, for a given needle diameter. As described in further detail below, the needle driver device is sized to be particularly suited for minimally invasive application, such that the needle driver can be inserted into a patient within a natural orifice or a small incision in a body wall and manipulated within the body.

Such needle driver devices may include a needle driver link configured to transmit rotational motion of a drive component to the needle to rotate the needle around a needle track to carry out a suturing procedure. The needle driver link may include a needle engagement portion configured to alternately engage and disengage the needle so as to drive rotational motion of the needle. The needle driver link may include various features configured to promote longevity and reliability of the needle driver device. For example, in some embodiments, the needle driver link can include a widened proximal end portion width to facilitate use of a relatively large bearing surface at the location at which drive forces are transmitted to the needle driver link. In addition, a distal end portion of the needle driver link may have a narrower width than the proximal end portion. In some embodiments, the needle driver link may have a tapered shape that tapers (e.g., narrows) in width towards the distal end. To permit the widened size of the proximal end portion width without a corresponding increase in the overall size (e.g., width or diameter) of the instrument, an orientation of the needle driver link as it moves may be constrained such that a longitudinal axis of the needle driver link is not always parallel with a longitudinal axis of the instrument itself. Embodiments of needle driver devices according to the present disclosure provide this increased robustness while maintaining an overall size that enables the needle driver to be used with, for example, an 8 mm cannula/instrument system. Other instrument sizes, such as 12 mm or 14 mm, or other sizes greater or lesser than 8 mm without limitation are within the scope of the disclosure.

A first portion of the needle driver link may be rotatably coupled to a first drive member, such as a rotary drive member that is rotatable to actuate movement of the needle driver link. A second portion, such as an intermediate portion of the needle driver link may be rotatably coupled to a second drive member, and the first drive member and the second drive member may rotate generally in a same rotational direction as the needle driver link is articulated to drive a needle around the needle track. The first drive member and second drive member may be mechanically coupled to rotate together by, for example, a gear train, a timing belt, or another mechanical coupling device. Due in part to the tapered shape of the needle driver link, the first drive member and second drive member may be rotationally coupled to the needle driver link at different radii from respective rotational axes of the first drive member and the second drive member. Thus, during a stroke of the driver link, the rotation of the first drive member and the second drive member may be in a ratio of other than 1:1, i.e., a non-unity ratio.

Due to the various kinematic connections and configurations of the first drive member, second drive member, and needle driver link, the distal end portion of the needle driver link may move along a non-circular arc path (e.g., tracing an arc that deviates from a perfect circular arcuate path). However, the needle and needle track may follow a generally circular arc path. Thus, the path followed by the distal end portion of the needle driver link and the path of the needle track may be non-coincident. A maximum deviation of the distal end portion of the needle driver link from the arc of the needle and needle track may be a distance less than a fraction of a cross-sectional width (e.g., half a cross-sectional diameter) of the needle, to ensure that the distal end portion of the needle driver link does not inadvertently disengage from the needle during operation. For example, according to some embodiments of the disclosure, the maximum deviation of the distal end portion of the needle driver link from the arc of the needle and needle track can be equal to or less than half of the cross-sectional diameter of the needle.

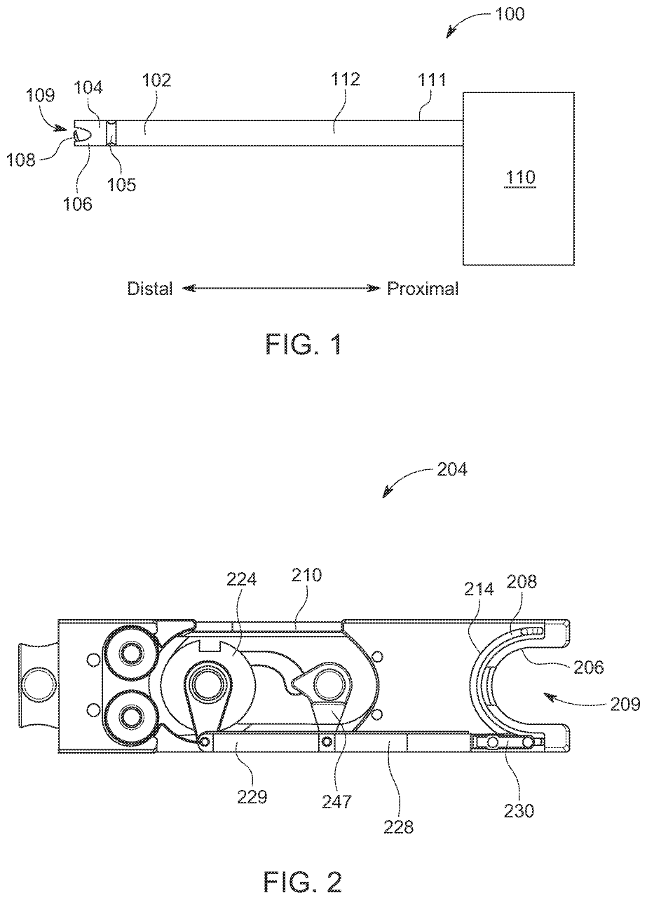

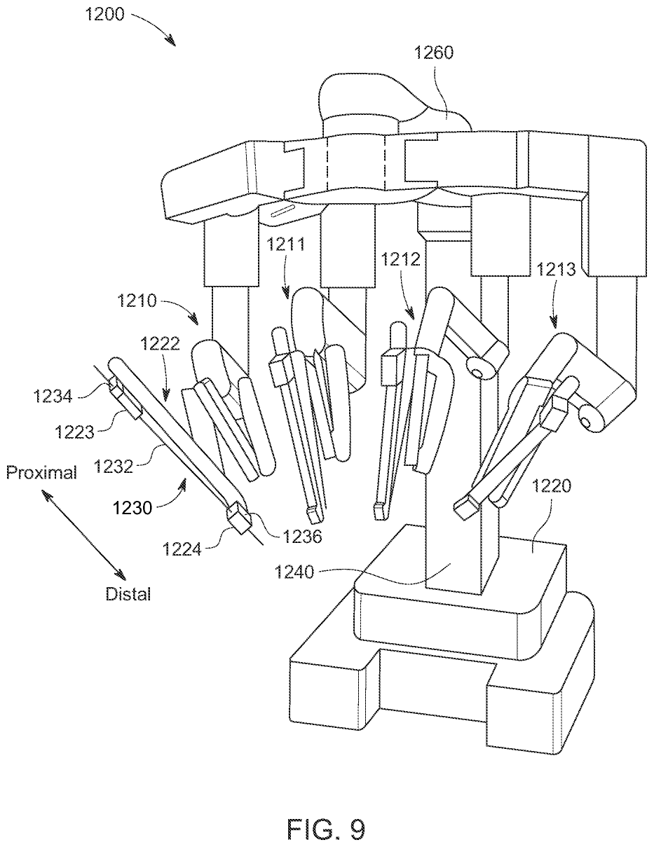

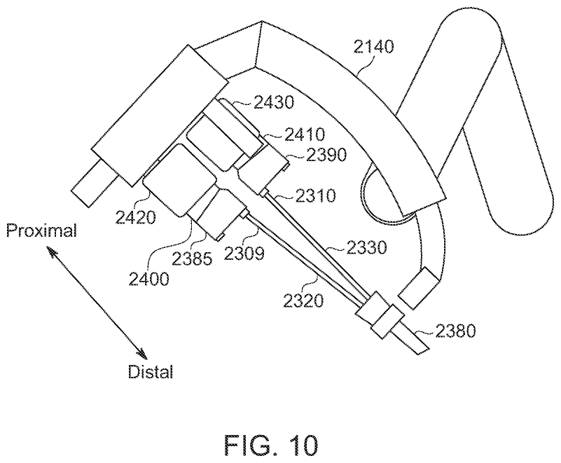

Referring now to FIG. 1, a schematic, side view of a needle driver device 100 according to some embodiments of the disclosure is shown. The needle driver device 100 includes an end effector portion 104, a shaft 112, and a transmission mechanism 110. The end effector portion 104 is located at a distal end portion 102 of the shaft 112. The end effector portion 104 comprises an arcuate-shaped distal end portion 106 (e.g., generally C-shaped and following a circular arc), which is configured to receive and house a curved needle 108 (e.g., also generally C-shaped, a sharpened tip portion of which is illustrated). In some embodiments, the needle 108 may be removable from the distal end portion 106 (e.g., for cleaning and sterilization). In some embodiments, the needle 108 has a curvature corresponding generally to the arc of the arcuate-shaped portion (e.g., generally C-shaped) of the end effector portion 104. The transmission mechanism 110 is coupled to a proximal end portion 111 of the shaft 112. The transmission mechanism 110 can be operably coupled with a computer-controlled (e.g., teleoperated) surgical manipulator system, such as the manipulator systems described in further detail below in connection with FIGS. 9 and 10, and/or the transmission mechanism 110 can be manually controlled with manually operated (e.g., handheld) actuators (not shown). The end effector portion 104 can optionally be coupled to the shaft 112 by a joint structure 105, such as a wrist, imparting one or more degrees of freedom to the end effector portion 104 relative to the shaft 112.

Drive inputs received at the transmission mechanism 110, whether through manual actuation or via a manipulator system, can actuate the end effector portion 104, such as by driving the needle 108 around a path defined partly by the arcuate-shaped distal end portion 106. The arcuate-shaped distal end portion 106 can face a distal direction of the end effector portion 104 and can define an opening or aperture 109, which may serve as a tissue gap for suturing tissue. Movement of the needle 108 across the aperture 109 of the arcuate-shaped distal end portion 106 can be used to, for example, suture tissue or other materials positioned within the aperture 109 of the distal end portion 106. For example, the needle 108 may have a sharp or pointed leading portion that is configured to penetrate tissue or other material positioned in the aperture 109. In some example embodiments, the arcuate-shaped distal end portion 106 includes an arcuate-shaped needle track, as discussed further below, exhibiting a radius of curvature similar to a radius of curvature of the needle 108, and the needle 108 rotates about a center of curvature of the arcuate-shaped track.

The transmission mechanism 110 can include one or more drive components configured to receive an oscillating, rotational drive input from a drive mechanism (such as a drive assembly of a surgical manipulator, e.g., drive assembly 1223 discussed in connection with FIG. 9), and to transmit the rotational drive input through one or more actuation members, such as, for example, a cable or rod (not shown) located within the shaft 112 and coupled at opposite ends to the transmission mechanism 110 and the end effector portion 104. In some embodiments, the actuation members can be cable drive elements having a pull/pull configuration. That is, the actuation member comprises a looped cable or two cables that can alternatingly be tensioned to achieve the desired drive motion. Various components within the end effector portion 104 can releasably engage the needle 108 to drive the needle 108 around portions of its arcuate path, as discussed in connection with FIGS. 2-8.

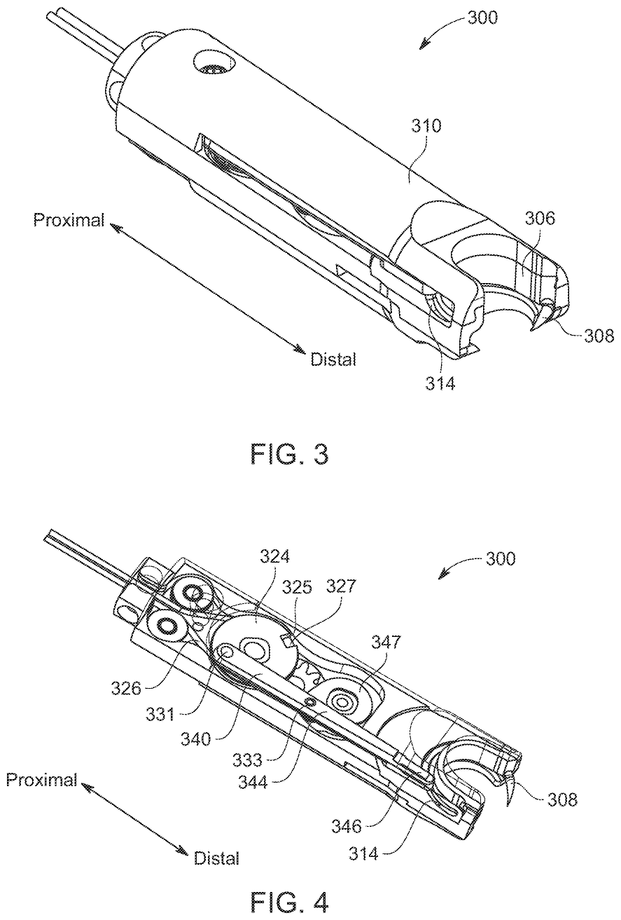

Referring now to FIG. 2, an end effector portion 204 of a needle driver device including a drive system is shown. The exemplary device of FIG. 2 can be used with, for example, the needle driver devices of FIG. 1 or 3. The needle driver end effector portion 204 comprise an arcuate-shaped distal end portion 206, which as described above can be a generally circular arc (e.g., C-shaped), defining an aperture 209. A curved needle 208, generally as described above in connection with FIG. 1, is configured to be received and positioned in a needle track 214 of the end effector portion 204. In the view of FIG. 2, the end effector portion 204 comprises a housing portion 210 that encloses various drive components.

The needle 208 is configured to rotate around the needle track 214 to perform a suturing procedure. As the needle 208 is driven around the track 214, the needle 208 moves through a first open end of the track 214, through the aperture 209, and returns back through a second open end of the track 214. The needle 208 may have a sharp or pointed leading portion that is configured to penetrate tissue or other material positioned in the aperture 209 as the needle 208 traverses the tissue or other material. Each complete rotation (i.e., 360-degree rotation) of the needle 208 around the needle track 214 can complete a single suture stitch in the tissue or other material positioned in the aperture 209 of the arcuate-shaped portion 206 of the needle driver device. In example embodiments, the thread of suturing material can be coupled (e.g., crimped) at a leading or trailing portion of the needle 208, and the thread can follow the needle 208 around the needle track 214 and through tissue or other material as the needle 208 rotates to complete each suture.

Needle driver devices according to various embodiments of the present disclosure include drive components to rotate the needle through a full rotation without mechanical drive components entering a suturing area defined by the aperture of the arcuate-shaped portion. As described further below, such needle driver devices include various components that receive input from the transmission mechanism (e.g., transmission mechanism 110 shown in FIG. 1) to actuate movement of the needle. In various embodiments, the transmission mechanism is coupled to a rotary drive mechanism, such as a pulley, in the distal end portion of the needle driver device via an actuation member. The actuation member can be or include, for example, one or more cables, belts, or other elements extending from the transmission mechanism, through the shaft, to the distal end portion of the needle driver device. Additional drive components convert oscillating rotational movement of the rotary drive mechanism into a reciprocating and curvilinear translational movement of another drive component along a desired path. These drive components can include features that removably couple with the needle such that the reciprocating movement of a drive components removably coupled with the needle drives the needle to move along the arcuate-shaped track and aperture of the arcuate-shaped distal end portion of the end effector, e.g., along a generally circular path.

For example, with continued reference to FIG. 2, the drive system of the needle driver device includes a needle driver link 228, a first drive member 224, and a second drive member 247. The first and second drive members can be configured to rotate about respective axes and can be referred to herein as rotary drive members. The needle driver link 228 extends generally distally from a proximal end portion 229 and terminates at a free, distal end portion 230 of the needle driver link 228. The first drive member 224 and the second drive member 247 are each coupled to the needle driver link 228 to drive the needle driver link 228 around the track 214. The first drive member 224 is coupled to a first portion of the needle driver link 228. In some embodiments, the first portion may be located at the proximal end portion 229 of the needle driver link 228. In other embodiments, the first portion may be located at an intermediate portion of the needle driver link 228 that is between the proximal end portion 229 and the distal end portion 230 of the needle driver link 228. The second drive member 247 is coupled to a second portion of the needle driver link 228, where the second portion is located at an intermediate portion of the needle driver link 228 that is distal to the coupling location of the first drive member 224 with needle driver link 228 and proximal to the distal end portion 230 of the needle driver link 228. The needle driver link 228 releasably engages with the needle 208 at the distal end portion 230 of the needle driver link 228 to drive the needle 208 around a 360-degree path. Oscillating rotary drive movement of the first drive member 224 and second drive member 247, both of which rotate about parallel axes, actuates movement of the needle driver link 228 in curvilinear translation such that the distal end portion 230 of the needle driver link 228 oscillates along the length of the needle track 214. The needle driver link 228 oscillates in alternating clockwise and counterclockwise rotations around the needle track 214 within the housing portion 210 (i.e., without the needle driver link 228 being inserted into and crossing the aperture 209). The needle driver link 228 alternatingly engages and releases the needle 208 to drive the needle around the needle track 214 and through the aperture 209 to insert sutures (e.g., by alternatingly coupling the needle driver link 228 to a first portion of the needle 208 near the leading portion of the needle 208 and a second portion of the needle near the trailing portion of the needle 208).

According to embodiments of the present disclosure, needle driver devices include driver links and associated components with features and configurations that facilitate use of stronger, more robust components without increasing the overall size (e.g., width or diameter) of the needle driver device. For example, needle driver devices of the present disclosure can include needle driver links with a tapered profile towards the distal end, needle driver links that follow a modified path in which the needle driver link deviates from a circular path, and other features. For example, devices of the present disclosure can include needle driver links that move in a combination of rotation and curvilinear translation and assume various non-parallel configurations throughout its range of motion during operation.

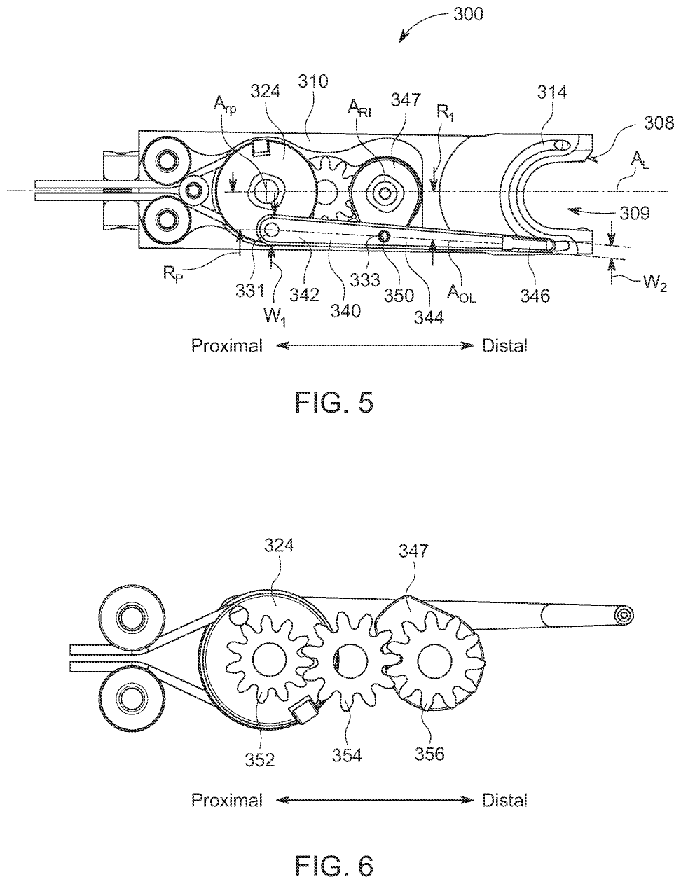

Referring now to FIGS. 3-5, a needle driver device 300 according to some embodiments of the disclosure is shown. The needle driver device 300 includes various features similar to needle driver devices described above, including an arcuate-shaped (e.g., C-shaped) distal end portion 306 and an arcuate-shaped needle track 314 configured to receive and drive a curved needle 308. The needle driver device 300 also includes a housing portion 310 that encloses various drive components of the needle driver device 300. FIG. 4 shows the needle driver device 300 of FIG. 3 with portions of the housing portion 310 omitted to show the various drive components and features of the needle driver device 300. The needle driver device 300 includes a rotatable first drive member 324 around which a cable actuation member 326 extends. As used herein, the first drive member 324 can be optionally referred to as a rotary drive member, such as in embodiments in which an actuation member is operably coupled to the first drive member 324 to actuate rotation of the first drive member 324. The actuation member 326 includes a cable stop 327 to prevent slippage of the actuation member 326 relative to the first drive member 324. A needle driver link 340 is coupled to the first drive member 324 by a first joint 331 at a first portion of the needle driver link 340. In some embodiments, the first portion may be located at a proximal end portion 342 of the needle driver link 340. In other embodiments, the first portion may be located at an intermediate portion of the needle driver link 340 that is between the proximal end portion 342 and a distal end portion 346 of the needle driver link 340. Similar to the embodiment of FIG. 2, the needle driver link 340 is also coupled to a rotatable second drive member 347 at a second portion 344 of the needle driver link 340 by a second joint 333, where the second portion 344 is located at an intermediate portion of the needle driver link 340 that is distal to the coupling location of the first drive member 324 with the needle driver link 340 and proximal to the distal end portion 346 of the needle driver link 228. First joint 331 may be a joint that allows relative rotation between the needle driver link 340 and the first drive member 324 (e.g., via a pivot joint such as a pin joint). As described in further detail below, second joint 333 may be a joint that allows relative rotation and constrained translation between the needle driver link 340 and the second drive member 347 as described further below (e.g., via a pivot joint such as a pin joint and a movement within a recess or slot). The first drive member 324 is rotatable about a first drive member axis A_{RP} (FIG. 5), and the second drive member 347 is rotatable about a second drive member axis A_{RI} (FIG. 5). Axes A_{RP} and A_{RI} are parallel to one another and intersect with and are perpendicular to a longitudinal axis A_{L} (FIG. 5) of the needle driver device 300. The distal end portion 346 of the needle driver link 340 includes one or more features configured to engage one or more detents or other features on the needle 308 to removably engage with and drive the needle 308 around a needle track 314 in response to the reciprocating motion of the needle driver link 340, similarly to that described above with respect to FIG. 2. Additional details regarding engagement features of the needle can be found in, for example, U.S. patent application Ser. No. 17/118,746 [UNPUBLISHED—ATTORNEY DOCKET NO. P06222-US] (filed Dec. 11, 2020) titled NEEDLE DRIVER DEVICES AND RELATED SYSTEMS AND METHODS.

Referring now to FIG. 5, the needle driver device 300 of FIGS. 3 and 4 is shown in top view with the housing portion 310 (FIG. 3) omitted as in FIG. 4. The needle driver device 300 has various features to provide reliable and robust drive components of the needle driver device 300. As shown in FIG. 5, the needle driver link 340 tapers in width from a first width W₁ at the proximal end portion 342 to a second width W₂, smaller than the first width, at the distal end portion 346. Due to the tapered shape of the needle driver link 340, and to avoid interference between the proximal end portion 342 and the housing portion 310, the center line A_{DL} of the needle driver link 340 is positioned further inboard at the proximal end portion 342 than it is at the distal end portion 346 of the needle driver link 340, relative to the width of the housing portion 310. In addition, the center line A_{DL} is positioned closer to a longitudinal axis A_{L} of the needle driver device 300 at the proximal end portion 342 than it is at the distal end portion 346 of the needle driver link 340. This relatively inboard positioning of the proximal end portion 342 relative to the width of the housing portion 310 can be achieved by reducing a radial distance R_{P} from the axis of rotation A_{RP} of the first drive member 324 to a center of the first joint 331 relative to a radial distance R_{I} from the axis of rotation A_{RI} of the second drive member 347 to a center of the second joint 333. As described above with reference to the embodiment of FIG. 2, the axis of rotation A_{RD} and the axis of rotation A_{RI} are parallel to each other and both are perpendicular to and intersect the longitudinal axis A_{L} of the needle driver device 300, and the axes of rotation A_{RP} and A_{RI} are parallel to one another. In other words, the center points about which the first and second drive members rotate are collinear on the longitudinal axis A_{L}. Thus, in this configuration, a maximum distance from the longitudinal axis A_{L} of the needle driver device 300 to the center of the second joint 333 is greater than a maximum distance from the longitudinal axis A_{L} of the needle driver device 300 to the center of the first joint 331. In other words, the first portion of the needle driver link 340 is rotatably coupled to the first drive member 324 at a location offset from the axis of rotation A_{RP} of the first drive member 324 by a first distance, and the second portion of the needle driver link 340 is rotatable coupled to the second drive member 347 at a location offset from the axis of rotation A_{RI} of the second drive member 347 by a second distance different from the first distance. However, in some embodiments, the center points about which the first and second drive members 324, 347 rotate are not collinear with the longitudinal axis A_{L}. The difference in radial distances R_{P} and R_{I} enables the distal end portion 346 of the needle driver link 340 to reach the ends of the needle track 314 to engage and disengage the needle 308. That is, the arc path traveled by the distal end portion 346 is larger than the arc path traveled by the proximal end portion 342, thereby enabling the distal end portion 346 to traverse along and reach the ends of the needle track 314 to properly engage the needle 308 during cyclical movement of the needle driver link 340 in spite of the relatively smaller arc path followed by the proximal end portion 342 as a result of the smaller radial distance R_{P}. While FIG. 5 depicts the taper in width as extending from the proximal end portion 342, in alternative embodiments, the needle driver link 340 may begin to taper in width from the first width W₁ at an intermediate portion of the needle driver link 340, wherein the intermediate portion is between the proximal end portion 342 and a distal end portion 346 of the needle driver link 340. For example, the needle driver link 340 may begin to taper in width at a position that is distal to a position where the first drive member 324 couples to the needle driver link 340. In another example, the needle driver link 340 may begin to taper in width at a position that is proximal to a position where the first drive member 324 couples to the needle driver link 340, and the needle driver link 340 may have a non-tapering portion that extends further proximally. In yet other examples, the needle driver link 340 may begin to taper in width at a position that is at or near to a position where the first drive member 324 couples to the needle driver link 340, and the needle driver link 340 may have a non-tapering portion that extends further proximally.

Due to the constraints that cause the distal end portion 346 and proximal end portion 342 to move along unequal path lengths, the needle driver link 340 moves in a combination of curvilinear translation and rotation as the first drive member 324 and second drive member 347 rotate and the distal end portion moves along the needle track 314 in response to rotation of the first drive member 324.

Because the proximal end portion 342, intermediate portion 344, and distal end portion 346 of the driver link 340 each follow paths having unequal path lengths, an additional degree of freedom can be provided in the interaction between the second drive member 347, first drive member 324, and the needle driver link 340 to enable the needle driver link 340 to move along its path of travel. For example, an additional degree of freedom can be provided by additional tolerance in the ability of the driver link 340 to move relative to the second joint 333. For example, referring again to FIG. 3, the second joint 333 comprises a pin joint, and the pin is received in a slot 350 in the driver link 340 that is slightly elongated along the longitudinal axis A_{DL} of the needle driver link 340. The slot 350 receives the pin of the second joint 333 and permits a small amount of movement of the pin along the longitudinal axis A_{DL} of the needle driver link 340 to prevent binding at the second joint 333.

While the elongated slot 350 in the needle driver link 340 provides an additional degree of freedom that can be helpful for the kinematic design of FIG. 3, alternative configurations could include an additional degree of freedom of movement between the first joint 331 and the needle driver link 340, and/or between the second joint 333 and the second drive member 347, the first joint 331 and the first drive member 324, or any other arrangement that provides one or more additional degrees of freedom of movement between the first drive member 324, the second drive member 347, and the needle driver link 340. Moreover, different arrangements for configuration of the first and second joints are contemplated, such as the pin of the joint being coupled to the needle driver link 340 and an elongated or otherwise enlarged slot provided in the first drive member 324 or second drive member 347 as applicable to provide the additional degree(s) of freedom. Such arrangements can ensure that the distal end portion 346 of the needle driver link 340 can move in the desired arcuate-shaped path without unduly binding motion of the overall needle driver link 340, which could lead to deformation or other damage to the needle driver link 340, or otherwise to undesired overall motion of the needle driver link 340.

The coupling of the first drive member 324 to the second drive member 347 via the needle driver link 340 causes the second drive member 347 to move in response to motion of the first drive member 324 and generally the motion of the first drive member 324 and the second drive member 347 are in tandem in that they rotate in the same direction. However, at the position at which distal end portion 346 of the needle driver link 340 is centered between ends of the needle track 314 and the longitudinal axis A_{DL} of the needle driver link 340 aligns with the longitudinal axis A_{L} of the needle driver device 300, the movement of the second drive member 347 becomes indeterminate (e.g., as a inflection point in the motion). That is, continued rotation of the first drive member 324 could inadvertently result in rotation of the second drive member 347 in the opposite direction of the first drive member 324, which, if allowed, could result in jamming or damage to internal components. To ensure the second drive member 347 and first drive member 324 always rotate in the same direction and the needle driver link 340 maintains its desired position, additional mechanical coupling components can be used to couple the movement of the first drive member 324 and the second drive member 347.

For example, in some embodiments, a gear train may additionally be used to couple the first drive member 324 and the second drive member 347. Referring now to FIG. 6, the drive system, including a gear train, of the needle driver device 300 is shown in isolation and from a side opposite to that shown in FIGS. 4 and 5. The first drive member 324 is coupled to a proximal drive gear 352, which is intermeshed with an idler gear 354. The idler gear 354 is in turn intermeshed with an intermediate drive gear 356 coupled to the second drive member 347. The idler gear 354 between the proximal drive gear 352 and the intermediate drive gear 356 ensures the first drive member 324 and second drive member 347 move in unison and prevents counter-rotation of the second drive member at the indeterminate position. While the embodiment of FIGS. 3-6 utilizes a gear train to constrain the desired motion of the second drive member 347 throughout rotation of the first drive member 324, other embodiments could include a timing belt or chain or other mechanical arrangements.

Due to the difference between the radial distance R_{D} of the first drive member 324's rotational axis A_{RP} to the first joint 331 and the radial distance RI of the second drive member 347's rotational axis A_{RI} to the second joint 333, the first drive member 324 sweeps over a larger arc (i.e., a larger proportion of a full circle) than the second drive member 347 as the first drive member 324 rotates to actuate the needle driver link 340 and drive the needle 308. Accordingly, in some embodiments, the gear train or other mechanical coupling between the first drive member 324 and second drive member 347 features a non-unity gear ratio. For example, in the embodiment of FIGS. 3-6, the ratio of the first drive member gear 352 to the second drive member gear 356 may be such that the second drive member 347 rotates over fewer degrees than the first drive member 324 for a given rotation of the first drive member 324.

In an example embodiment, the gear ratio of the proximal drive gear 352 to the intermediate drive gear 356 is 10:11. However, other gear ratios may be selected as desired and are considered within the scope of the disclosure. Further, the gear ratio (or pulley ratio, or ratio of other mechanical coupling) may or may not be exactly equal to a ratio of R_{D} to RI. In embodiments in which the gear ratio is not equal to the ratio of R_{D} to R_{I}, play in various components of the drive system can still allow for free movement of the components relative to one another. For example, the elongated slot 350 in the intermediate portion can provide enough play such that the gear ratio does not have to exactly match the ratio of R_{D} to RI to enable movement of the drive components along the desired paths without binding. Alternatively, or in addition, the gear train may be provided with a degree of backlash (e.g., tolerance between gear teeth) sufficient to enable free movement of the components without a precise match between the ratio of R_{D} to R_{I}.

During use, the first drive member 324 is actuated in an oscillating fashion in alternating rotational directions. The needle driver link 340, rotatably coupled at the first drive member 324 and at the second drive member 347, moves in generally in translation (with a small rotational component as discussed above) such that the distal end portion 346 of the needle driver link 340 travels from one end to the other of the needle track 314 generally following the arc of the needle track 314. In response to rotation of the first drive member 324 in a first direction (e.g., clockwise as viewed in FIG. 5), the distal end portion 346 of the needle driver link 340 travels to the opposite end of the needle track 314, drawing the needle 308 through the needle track 314 and through the aperture 309.

Once the needle driver link 340 reaches the opposite side of the needle track 314, i.e., so the needle driver link 340 is opposite the position shown in FIG. 5 (i.e., at the top of the drawing rather than the bottom), the distal end portion 346 disengaged from the needle 308. The first drive member 324 rotates back in a second direction opposite to the first direction (e.g., counterclockwise as viewed in FIG. 5) with the distal end portion 346 of the needle driver link 340 disengaged from the needle 308, bringing the needle driver link 340 back to the position shown in FIG. 5. Upon return of the needle driver link 340 to this position, the distal end portion 346 engages a leading portion of the needle, now positioned adjacent the distal end portion 346 at the bottom of the needle track 314. A subsequent rotation of the first drive member in the first direction moves the needle driver link 340 back along the needle track 314, pulling the needle 308 through the needle track 314 and back to the initial position of the needle 308 shown in FIG. 5. Finally, rotation of the first drive member 324 in the second direction causes the distal end portion to disengage the needle 308 and return to the configuration shown in FIG. 5, at which time the cycle can repeat. In this manner, the needle driver link 340 drives the needle 308 repeatedly through the aperture 309, and inserts suture material (not shown), which may be coupled to the needle 308, repeatedly through tissue to insert sutures. It should be appreciated that the first and second directions are provided by way of example, and in alternative embodiments, the first direction may be counterclockwise and the second direction may be clockwise.

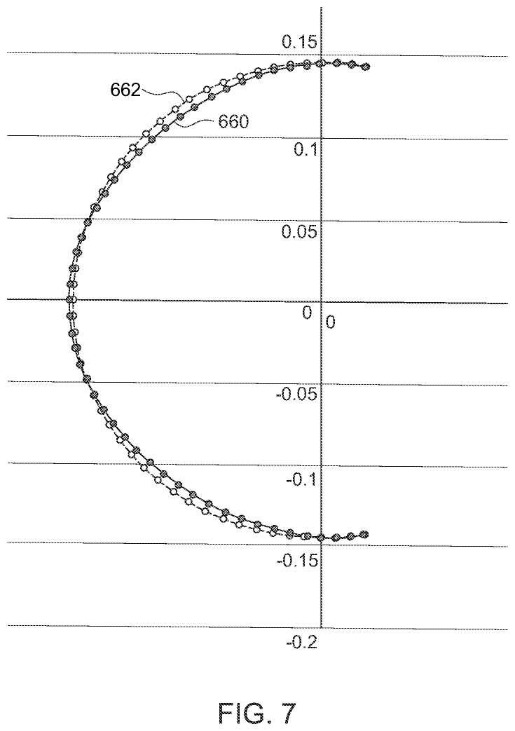

In the embodiment of FIGS. 4-6, the kinematic configuration of the first drive member, the second drive member, and the needle driver link results in the distal end portion of the needle driver link 340 following a non-circular arcuate path. That is, since the proximal end portion of the needle driver link 340 is constrained to follow an arc defined by radial distance R_{P} (the arc traced by the first joint 331 as the first drive member 324 rotates), constraining the distal end portion 346 of the needle driver link 340 to follow an arc larger than the arc of the proximal end portion 342 at the first joint 331 (e.g., constraining the distal end portion 346 of the needle driver link 340 to follow generally the arcuate path (e.g., circular arc) of the needle 308) would result in an over-constrained system. FIG. 7 shows a graphical diagram of the movement of the distal end portion 346 of the needle driver link 340, represented by path 660, compared to the arc traveled by the needle 308 along the needle track 314, represented by needle path 662. As is apparent from FIG. 7, the path 660 of the distal end portion 346 of the needle driver link 340 and the needle path 662 of the needle 308 are not coincident.

Deviation from the needle path 662 by the distal end portion 346 can be acceptable as long as the distal end portion 346 follows the needle path 662 sufficiently close so as to remain engaged with the needle 308. Stated another way, the path of the distal end portion 346 of the needle driver link 340 may be adjacent to, but not coincident with the path of the needle path 662. The permissible deviation from the needle path 662 depends on characteristics of the needle 308, such as a cross-sectional thickness (e.g., cross-sectional diameter) and the particular interface of the distal end portion 346 with the needle 308. For example, for a given needle cross-sectional size and shape, too much deviation of the distal end portion 346 from the needle track 314 could result in distal end portion 346 of the needle driver link 340 becoming disengaged from the needle 308.

In some embodiments, by way of example, the needle has a thickness (e.g., cross-sectional diameter) of about 0.028 inches to 0.032 inches (about 0.71 mm to 0.81 mm). Deviation of the distal end of the needle driver link from the needle path of less than half the thickness of the needle may enable the desired dimensions and kinematic relationships of the drive components while maintaining engagement of the distal end portion of the needle driver link with the needle 308 throughout the range of motion of the needle driver link. In the embodiment of FIGS. 3-6, the maximum deviation of the distal end portion of the needle driver link from the path of the needle 308 along any point of the distal end portion's path is about 0.005″ (0.13 mm) or less. In other embodiments, the maximum deviation can be less than or greater than 0.005″ to the extent that the distal end portion remains engaged with the needle 308 throughout the range of motion of the distal end portion despite the deviation. The dimensions noted above are by way of example only, and the cross-sectional size and shape of the needle and the amount of deviation of the distal end portion 346 from the needle track 314 can differ among various embodiments. An example range of needle cross-sectional diameters can be from about 0.01″ (0.254 mm) and 0.04″ (1.02 mm) and the deviation from the track can be from about 0.002″ (0.0508 mm) and 0.01″ (0.254 mm). These dimensions are provided as examples only, and dimensions falling outside of the listed ranges are still within the scope of the disclosure.

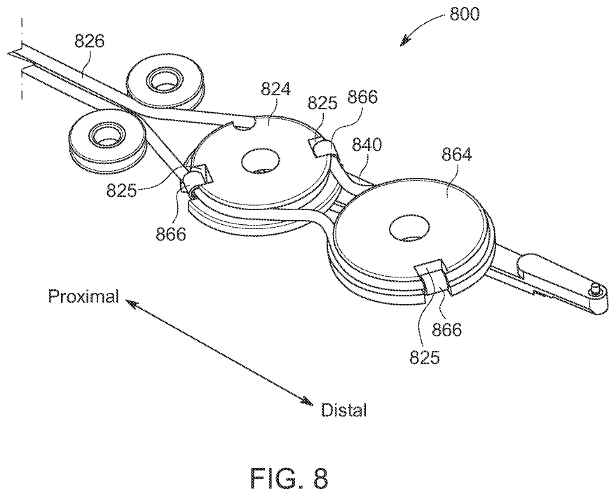

Referring now to FIG. 8, a drive system 800 for a needle drive device according to some embodiments is shown in isolation for simplicity. The drive system 800 can be used with, for example, the needle driver devices of FIG. 1 or 3. The drive system 800 includes a first drive member 824, a second drive member 864, and a needle driver link 840 coupled to the first drive member 824 and second drive member 864 by joints similar to first and second joints 331, 333 discussed in connection with the embodiments of FIGS. 3-6. A pull-pull cable actuation member 826 is routed around both the first drive member 824 and the second drive member 864 and passes through cable stops 866 that are received in notches 825 in the first drive member 824 and second drive member 864. The cable stops maintain the rotational relationship between the first drive member 824 and second drive member 864 and prevent the actuation member 826 from slipping relative to the first drive member 824 and second drive member 864. In the embodiment of FIG. 8, the first drive member 824 and second drive member 864 are rotationally coupled by the actuation member 826 instead of through the use of a gear train discussed above in connection with the embodiments of FIGS. 3-6. As in the embodiments of FIGS. 3-6, the first drive member 824 and second drive member 864 may sweep over different proportions of a full 360 degree arc due to differences in radial distances from axes of rotation of the first drive member and second drive member to respective joints (e.g., pin joints) that couple the needle driver link 340 to the first drive member 824 and second drive member 864. The first drive member 824 and second drive member 864 may also have different overall radii such that actuation of the actuation member 826 rotates each of the first drive member 824 and second drive member 864 at different rates as appropriate for the differences in radii of the joints relative to the axes of rotation of each of the first and second drive members 824, 864.

Embodiments of needle driver devices discussed above and otherwise according to the present disclosure can provide mechanical robustness to promote longevity of service and reliability, particularly by increasing the bearing area at locations where drive forces are concentrated. Further, embodiments of needle driver devices according to the present disclosure provide this increased robustness while maintaining an overall size that enables the needle driver to be used with, for example, an 8 mm cannula/instrument system, 12 mm cannula instrument system, 14 mm cannula/instrument system, or other size systems.

Embodiments described herein may be used, for example, with remotely operated, computer-assisted systems (such, for example, teleoperated surgical systems) such as those described in, for example, U.S. Pat. No. 9,358,074 (filed May 31, 2013) to Schena et al., entitled “Multi-Port Surgical Robotic System Architecture”, U.S. Pat. No. 9,295,524 (filed May 31, 2013) to Schena et al., entitled “Redundant Axis and Degree of Freedom for Hardware-Constrained Remote Center Robotic Manipulator”, and U.S. Pat. No. 8,852,208 (filed Aug. 12, 2010) to Gomez et al., entitled “Surgical System Instrument Mounting”, each of which is hereby incorporated by reference in its entirety. Further, embodiments described herein may be used, for example, with a da Vinci® Surgical System, such as the da Vinci Si® Surgical System, da Vinci X® Surgical System, the da Vinci Xi® Surgical System, all with or without Single-Site® single orifice surgery technology, or the daVinci SPR Surgical System, all commercialized by Intuitive Surgical, Inc., of Sunnyvale, California.

The embodiments described herein are not limited to the surgical systems noted above, and various other teleoperated, computer-assisted surgical system configurations may be used with the embodiments described herein. Further, although various embodiments described herein are discussed in connection with a manipulating system of a teleoperated surgical system, the present disclosure is not limited to use with a teleoperated surgical system. Various embodiments described herein can optionally be used in conjunction with hand-held, manual instruments.

As discussed above, in accordance with various embodiments, surgical instruments of the present disclosure are configured for use in teleoperated, computer-assisted surgical systems employing robotic technology (sometimes referred to as robotic surgical systems). Referring now to FIG. 9, an embodiment of a manipulator system 1200 of a computer-assisted surgical system, to which surgical instruments are configured to be mounted for use, is shown. Such a surgical system may further include a user control system, such as a surgeon console (not shown) for receiving input from a user to control instruments coupled to the manipulator system 1200, as well as an auxiliary system, such as auxiliary systems associated with the da Vinci® systems noted above.

As shown in the embodiment of FIG. 9, a manipulator system 1200 includes a base 1220, a main column 1240, and a main boom 1260 connected to main column 1240. Manipulator system 1200 also includes a plurality of manipulator arms 1210, 1211, 1212, 1213, which are each connected to main boom 1260. Manipulator arms 1210, 1211, 1212, 1213 each include an instrument mount portion 1222 to which an instrument 1230 may be mounted, which is illustrated as being attached to manipulator arm 1210. While the manipulator system 1200 of FIG. 9 is shown and described having a main boom 1260 to which the plurality of manipulator arms are coupled and supported thereby, in other embodiments, the plurality of manipulator arms can be coupled and supported by other structures, such as an operating table, a ceiling, wall, or floor of an operating room, etc.

Instrument mount portion 1222 comprises a drive assembly 1223 and a cannula mount 1224, with a transmission mechanism 1234 (which may generally correspond to the transmission mechanism 110 discussed in connection with FIG. 1) of the instrument 1230 connecting with the drive assembly 1223, according to an embodiment. Cannula mount 1224 is configured to hold a cannula 1236 through which a shaft 1232 of instrument 1230 may extend to a surgery site during a surgical procedure. Drive assembly 1223 contains a variety of drive and other mechanisms that are controlled to respond to input commands at the surgeon console and transmit forces to the transmission mechanism 1234 to actuate the instrument 1230. Although the embodiment of FIG. 9 shows an instrument 1230 attached to only manipulator arm 1210 for ease of viewing, an instrument may be attached to any and each of manipulator arms 1210, 1211, 1212, 1213.

Other configurations of surgical systems, such as surgical systems configured for single-port surgery, are also contemplated. For example, with reference now to FIG. 10, a portion of an embodiment of a manipulator arm 2140 of a manipulator system with two surgical instruments 2309, 2310 in an installed position is shown. The surgical instruments 2309, 2310 can generally correspond to instruments discussed above, such as needle driver device 100 disclosed in connection with FIG. 1. For example, the embodiments described herein may be used with a DA VINCI SPR Surgical System, commercialized by Intuitive Surgical, Inc. of Sunnyvale, California. The schematic illustration of FIG. 10 depicts only two surgical instruments for simplicity, but more than two surgical instruments may be mounted in an installed position at a manipulator system as those having ordinary skill in the art are familiar with. Each surgical instrument 2309, 2310 includes a shaft 2320, 2330 that at a distal end has a moveable end effector or an endoscope, camera, or other sensing device, and may or may not include a wrist mechanism (not shown) to control the movement of the distal end.

In the embodiment of FIG. 10, the distal end portions of the surgical instruments 2309, 2310 are received through a single port structure 2380 to be introduced into the patient. As shown, the port structure includes a cannula and an instrument entry guide inserted into the cannula. Individual instruments are inserted into the entry guide to reach a surgical site.

Other configurations of manipulator systems that can be used in conjunction with the present disclosure can use several individual manipulator arms. In addition, individual manipulator arms may include a single instrument or a plurality of instruments. Further, as discussed above, an instrument may be a surgical instrument with an end effector or may be a camera instrument or other sensing instrument utilized during a surgical procedure to provide information, (e.g., visualization, electrophysiological activity, pressure, fluid flow, and/or other sensed data) of a remote surgical site.

Transmission mechanisms 2385, 2390 (which may generally correspond to transmission mechanism 110 disclosed in connection with FIG. 1) are disposed at a proximal end of each shaft 2320, 2330 and connect through a sterile adaptor 2400, 2410 with drive assemblies 2420, 2430. Drive assemblies 2420, 2430 contain a variety of internal mechanisms (not shown) that are controlled by a controller (e.g., at a control cart of a surgical system) to respond to input commands at a surgeon side console of a surgical system to transmit forces to the transmission mechanisms 2385, 2390 to actuate surgical instruments 2309, 2310.

The embodiments described herein are not limited to the embodiments of FIG. 9 and FIG. 10, and various other teleoperated, computer-assisted surgical system configurations may be used with the embodiments described herein. The diameter or diameters of an instrument shaft and end effector are generally selected according to the size of the cannula with which the instrument will be used and depending on the surgical procedures being performed.

This description and the accompanying drawings that illustrate various embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A needle driver device, comprising:
a needle track defining a curved path; and
a drive system configured to operably engage with and drive a needle along the needle track, the drive system comprising a first drive member, a second drive member, and a needle driver link,
wherein the first drive member is rotatable about a first drive member axis,
wherein the second drive member is rotatable about a second drive member axis spaced apart from the first drive member axis, and
wherein the needle driver link comprises:
a distal end portion removably engageable with a needle that is positionable in the needle track, the distal end portion being movable along an arcuate-shaped path proximate the needle track,
a first portion rotatably coupled to the first drive member at a location offset from the first drive member axis by a first distance, and
a second portion between the first portion and the distal end portion, the second portion rotatably coupled to the second drive member at a location offset from the second drive member axis by a second distance different from the first distance.

2. The needle driver device of claim 1, wherein the first drive member axis and the second drive member axis are parallel to each other and are perpendicular to and intersect a longitudinal axis of the needle driver device.

3. The needle driver device of claim 1, further comprising an actuation member operably coupled to one or both of the first drive member and the second drive member to transmit force to rotate the one or both of the first drive member and the second drive member.

4. The needle driver device of claim 3, wherein the actuation member comprises a cable.

5. The needle driver device of claim 1, wherein the first drive member and the second drive member are operatively coupled to one another such that the first drive member and the second drive member rotate in a same rotational direction throughout a range of motion of the first drive member and the second drive member.

6. The needle driver device of claim 5, further comprising a gear train mechanically coupling the first drive member and the second drive member.

7. The needle driver device of claim 6, wherein the gear train comprises a first gear coupled to the first drive member, an idler gear intermeshed with the first gear, and a third gear coupled to the second drive member and intermeshed with the idler gear.

8. The needle driver device of claim 5, wherein a ratio of rotation of the first drive member to rotation of the second drive member is not equal to one.

9. The needle driver device of claim 8, wherein a ratio of rotation of the first drive member to rotation of the second drive member is less than one.

10. The needle driver device of claim 1, wherein one or both of:
the first portion of the needle driver link is rotatably coupled to the first drive member at a location movable along a longitudinal axis of the needle driver link; and
the second portion of the needle driver link is rotatably coupled to the second drive member at a location movable along the longitudinal axis of the needle driver link.

11. The needle driver device of claim 1, wherein the needle track is arc-shaped.

12. The needle driver device of claim 11, wherein the needle track is shaped as a circular arc.

13. The needle driver device of claim 12, wherein the path along which distal end portion of the needle driver link is movable is a non-circular arc path.

14. The needle driver device of claim 13, further comprising a curved needle movable along the needle track.

15. (canceled)

16. The device of claim 1, wherein the needle driver link comprises a tapered shape tapering from a first width at the first portion to a second width smaller than the first width at the distal end portion.

17. A needle driver device comprising:
a curved needle track defining a curved path;
a rotary drive member; and
a needle driver link comprising:
a distal end portion removably engageable with a curved needle configured to be received in the curved needle track,
a first portion coupled to the rotary drive member; and
a longitudinal axis extending between the distal end portion and the first portion,
wherein the needle driver link moves in response to rotation of the rotary drive member to drive the curved needle along the curved needle track, and
wherein, in response to rotation of the rotary drive member, the distal end portion of the needle drive link traverses an arcuate-shaped path that deviates in shape from the curved path defined by the curved needle track.

18. The needle driver device of claim 17, wherein the rotary drive member is a first rotary drive member, and wherein the needle driver link comprises a second portion rotatably coupled to a second rotary drive member.

19. The needle driver device of claim 18, wherein the first rotary drive member and the second rotary drive member are coupled to rotate in a same direction.

20. The needle driver device of claim 19, wherein the first rotary drive member and the second rotary drive member are rotationally coupled by an actuation member actuatable to rotate the first rotary drive member and the second rotary drive member.

21. The device of claim 19, further comprising:
a gear train coupling the first rotary drive member and the second rotary drive member.

22.-24. (canceled)
